(19) United States
(12) Patent Application Publication
(10) Pub. No.: US 2009/0065009 A1
(43) Pub. Date: Mar. 12, 2009

(54) Method and tools for the reduction of excess skin

(76) Inventors: Geoffrey C. Gurtner, Stanford, CA (US); Kenneth N. Horne, San Francisco, CA (US)

Correspondence Address: HOEKENDIJK & LYNCH, LLP, P.O. BOX 4787, BURLINGAME, CA, 94011-4787, US

(21) Appl. No.: 12/229,067

(22) Filed: Aug. 18, 2008

Related U.S. Application Data
(60) Provisional application No. 60/966,089, filed on Aug. 23, 2007.

(51) Int. Cl. A61B 17/00

(52) U.S. Cl. 128/898; 606/151

(57) ABSTRACT

Methods and devices for removing excess skin are described. The excess skin is gathered into a bundle, which is mechanically unloaded relative to the skin around the bundle. The bundle of excess skin is maintained for an extended period of time, such as at least three weeks, to permit the body to absorb some of the excess tissue.

### BACKGROUND OF THE INVENTION

This invention is directed to the treatment and reduction of excess skin. Excess skin may occur, for example, after massive weight loss secondary to bariatric surgery to treat obesity.

### SUMMARY OF THE INVENTION

Unlike conventional approaches that surgically remove excess skin, the present invention takes advantage of the body's ability to reabsorb skin when the correct conditions are created. Details of the techniques to create those conditions vary depending on the part of the body being treated. Particular areas of interest include face, neck, thighs, arms, abdomen, breasts, buttocks, and the back. Without limitation, to illustrate the principles of the invention, the treatment of excess skin on the arms will be used as an example. Application of the technique to other parts of the body uses the same principles.

A small incision is created at the junction of the arm and the back slightly posterior to the axilla. A tool is inserted and is used to create space between the skin of the lower arm and the underlying fascia. The skin can now be drawn posteriorly and gathered in the vicinity of the axilla. This action draws the skin snugly over the arm. A support device is used to hold the excess skin in a bundle. The support device is designed to mechanically unload the skin providing the appropriate conditions for the body to reabsorb the excess tissue. The support device and bundle of excess skin is bandaged typically for two to six weeks over which time the excess skin is reabsorbed by the body. The skin that was stretched over the arm reattaches to the underlying structures leaving a firm, unscarred surface.

An advantage of the present invention is that the skin may be remodeled and excess skin removed without surgically removing the tissue. As such, the area having the excess skin is otherwise normal and healthy. The present invention provides a reduction in trauma and scarring compared to procedures which simply remove the excess skin. Removal of the excess skin is somewhat traumatic and necessarily requires forming an incision around the entire area which is to be removed which may lead to undesirable scarring.

In another embodiment of the present invention where excess skin is present but an incision is not made, the excess skin is drawn in to smaller sections, each mechanically unloading the skin in a similar fashion. Since underlying structures connecting the skin to the underlying fascia prevent drawing the excess skin all together it is necessary to collect the excess skin into smaller sections. Once collected, all bundles of excess skin are supported in a similar fashion for a similar duration.

### BRIEF DESCRIPTION OF DRAWINGS

FIG. 1 example of a person's arm after massive weight loss.

FIG. 2A shows an embodiment of the inventive tool.

FIG. 2B shows a balloon inflated on the tool.

FIG. 2C shows another dissection tool.

FIG. 3 shows the acute presentation after the inventive surgical procedure.

FIG. 4 shows embodiments of the inventive support device.

FIG. 5A shows another device for removing excess skin in a relaxed state.

FIG. 5B shows the device of FIG. 5A stretched to an expanded state.

FIG. 6A is a cross-sectional view of the device of FIG. 5A.

FIG. 6B is a cross-sectional view of the device of FIG. 5B.

FIG. 7A shows the device of FIG. 5B before attachment to the skin.

FIG. 7B shows the device adhered to the skin in the stretched state.

FIG. 7C shows the device holding a bundle of skin.

FIG. 8A shows another support device which may be worn around the waist.

FIG. 8B shows another support device.

FIG. 9A shows another device for removing excess skin which is cinched.

FIG. 9B shows the device of FIG. 9A cinched to isolate excess skin.

FIG. 10A shows still another device for removing excess skin.

FIG. 10B shows the device of FIG. 10A attached to the skin to isolate excess skin.

FIG. 11 shows another device for gathering excess skin.

FIG. 12A shows an area where excess skin has been identified.

FIG. 12B shows the device of FIG. 11 inserted into the skin around the area where the excess skin lies.

FIG. 12C shows the device forming a closed loop around the excess skin.

FIG. 12D shows the device of FIG. 11 collapsed into a bundle of excess skin.

### DETAILED DESCRIPTION

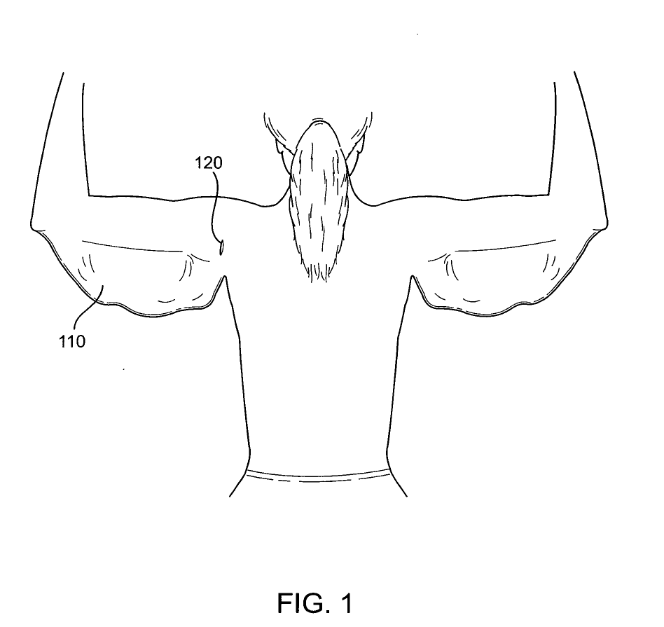

FIG. 1 shows an example of a person's arm after massive weight loss. The excess skin 110 hangs under the force of gravity. The present invention is directed to reducing the excess skin by inducing the body to absorb the excess skin. As will be described, the skin is gathered together into a bundle and secured in this state. The skin may be gathered and supported in a manner which mechanically unloads the skin which may be a condition which enhances the ability of the body to reabsorb the excess skin.

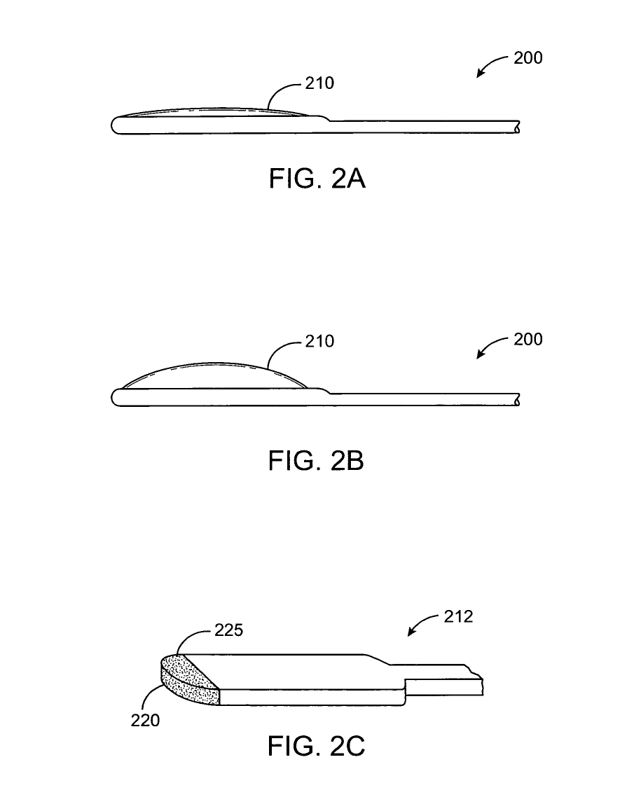

The skin may be separated from an underlying layer before being gathered into a bundle. To this end, an incision 120 may be created, for example, slightly posterior to the axilla when treating excess skin on the upper arm as shown in FIG. 1. The incision 120 is preferably small, but may be extended as needed. In this example, a tool 200 (shown in FIGS. 2A and 2B) is inserted into the incision 120 and is advanced using blunt dissection to create a plane between the skin with its subcutaneous fat and the underlying fascia. Alternatively the plane may be in the subcutaneous fat itself. The tool 200 may be navigated with or without endoscopy, fluoroscopy or other visualization technology. Electrosurgical techniques may be used to control bleeding if necessary.

FIG. 2A shows the tool having an integral dissection balloon 210. In FIG. 2A the balloon 210 is deflated. The leading edge 205 of the tool advances by blunt dissection, and a space is increased through the use of the balloon inflation as shown in FIG. 2B. FIG. 2C shows a tool 212 that can be advanced with ultrasonic assistance. The leading edge 220 incorporates an ultrasound transducer 225 that facilitates advancing the tool through fat. The skin may be separated from underlying layers using any suitable device such as a blade, cannula or needle.

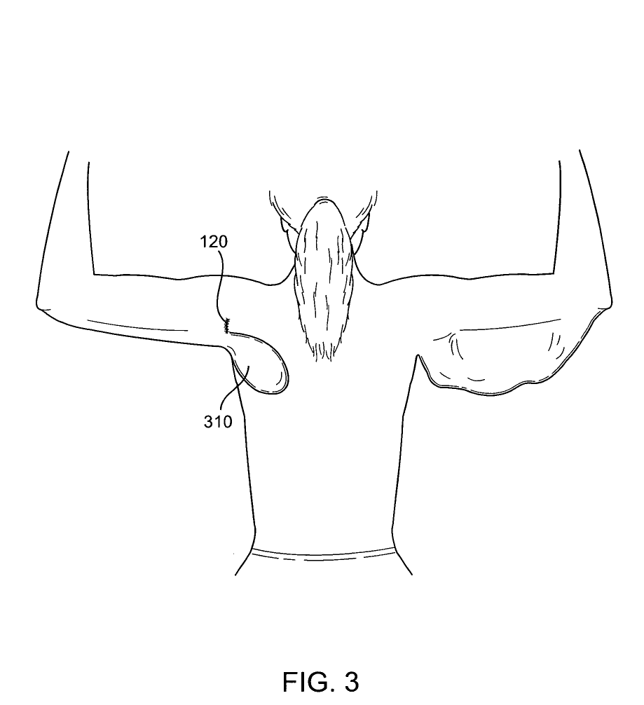

Once a plane has been created between the skin and the underlying fascia, the skin is drawn into a bundle 310 as shown in FIG. 3. At the surgeon's discretion, some trimming of the excess skin may occur in the vicinity of the initial incision 120. The incision 120 is then sutured closed. It can be appreciated that a relatively large amount of excess skin has been gathered into the bundle 310 as compared to the size of the incision. An advantage of the present invention is that surgical removal of the excess skin would require a much longer incision and may produce more visible scarring. Furthermore, the incision may also be positioned to lie within the bundle of excess skin and, as such, may produce a less noticeable scar. Of course, the incision may be separate from the excess skin as well as shown in FIG. 3. The present invention may also be practiced without the incision and dissection of the skin depending upon whether the skin may be adequately gathered without dissection.

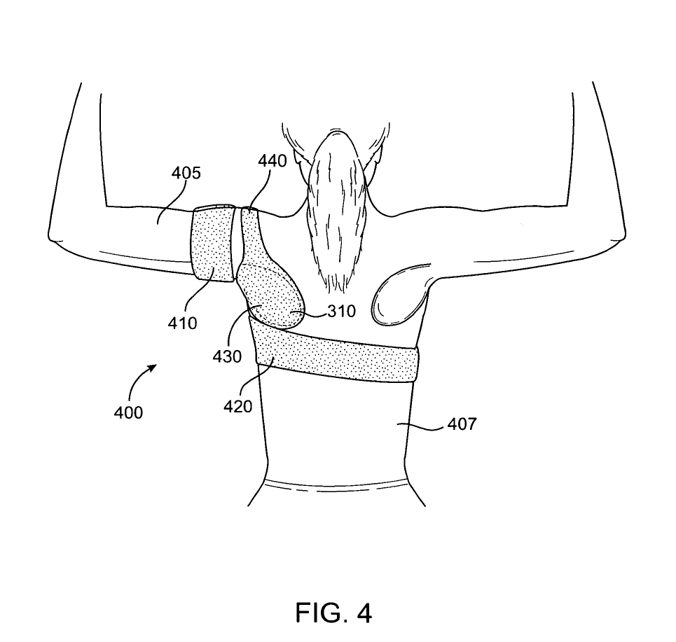

A support device 400 is used to hold the skin in the bundle 310 as shown in FIG. 4. The support 400 device may take a variety of forms. In this embodiment, the support device 400 has straps 410, 420, 440 and a pouch 430 made of fabric webbing. An arm strap 410 serves to maintain the patient's skin snug against the upper arm 405 and to anchor the support device 400 at one end. A torso strap 420 anchors the pouch 430 to the patient's torso 407. A shoulder strap 440 supports the pouch 430 against the force of gravity. The pouch 430 holds the bundled skin securely so it is mechanically unloaded, thereby creating the appropriate conditions for the body to reabsorb the excess skin. The straps 410, 420, 440 may be adjusted individually and relative to each other using fasteners to support the bundle of skin 310. The pouch 430 may be adapted to be adjustable, and/or may be available in a number of sizes to fit the particular patient. In some instances it may be advantageous to include adhesive components in the support device 400 to assure it is held securely to the patient. It is anticipated that bandages or a compression garment may be used in conjunction with the support device 400. The pouch 430 may be removable from the rest of the support device 400 so that the pouch 430 may be cleaned or changed as desired.

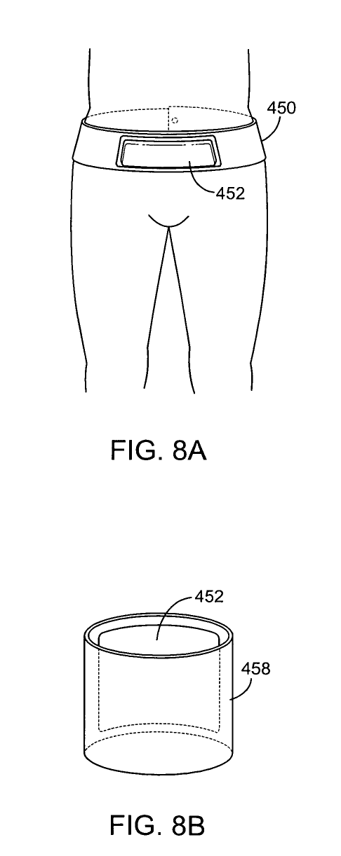

The present invention may be practiced in any area of the body. Referring to FIG. 8A, another support device 450 is shown which may be worn around the waist to reduce excess skin around the waistline and/or stomach. The support device 450 has a pouch 452 which holds the excess tissue. Referring to FIG. 8B, still another support device 456 is shown which may be worm on the arm or leg. The support device 456 has a pouch 458 which holds the excess skin which has been gathered together. It is understood that any of the devices described herein which are used to gather excess skin together may be used with any of the support garments for treating the stomach, waist, arms, legs, back or any other suitable area having excess skin.

The bundle 310 is maintained for an extended period of time so that the excess skin can be reabsorbed by the body. After a period of typically one to six weeks, the skin on the upper arm 405 reattaches to the underlying structures and a portion of the excess skin in the pouch 430 is reabsorbed by the body. When a reasonable amount of tissue has been gathered together, the bundle 310 may be held and maintained for at least three weeks to allow the body to absorb a portion of the excess skin.

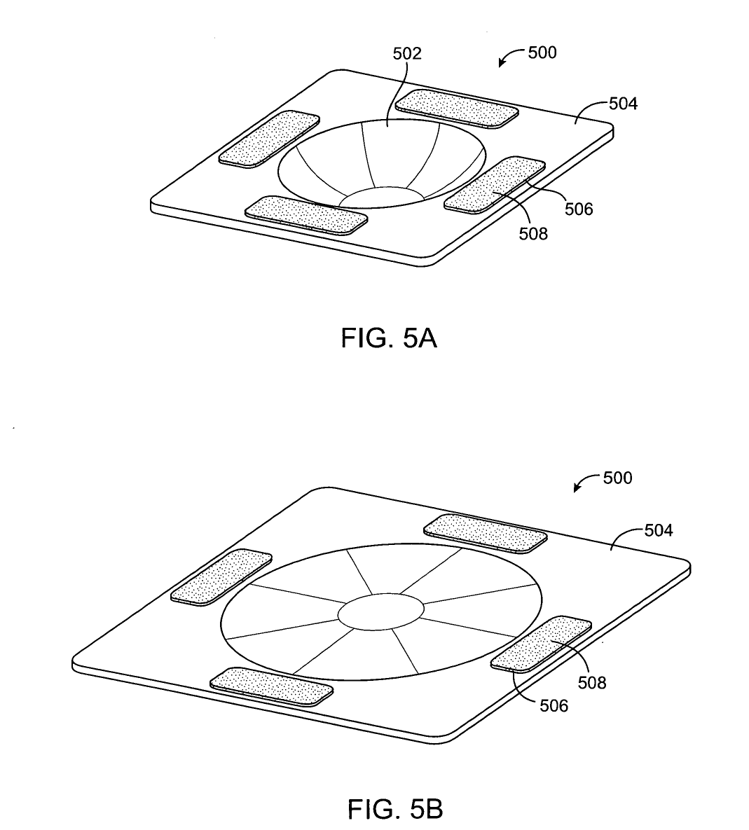

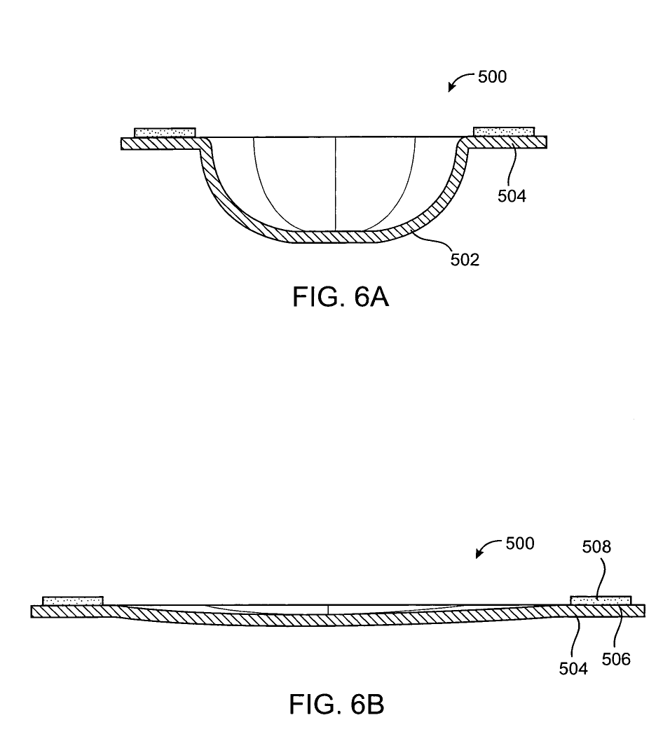

Referring to FIGS. 5A, 5B, 6A and 6B, another support device 500 is shown for holding a bundle of excess skin. This embodiment can be designed to create a plurality of pouches of mechanically unloaded skin, and is especially adaptable but not limited to uses where an incision is not made. The excess skin is held in a pouch 502 which is surrounded by an elastic membrane 504. The device 500 can be stretched from the relaxed state of FIG. 5A to the expanded or stretched state of FIG. 5B. The support device 500 includes adhesive portions 506 covered by removable strips 508 for adhering the device 500 to the skin. The device 500 may be attached to the skin in any other manner such as with barbs, sutures, hooks or glue.

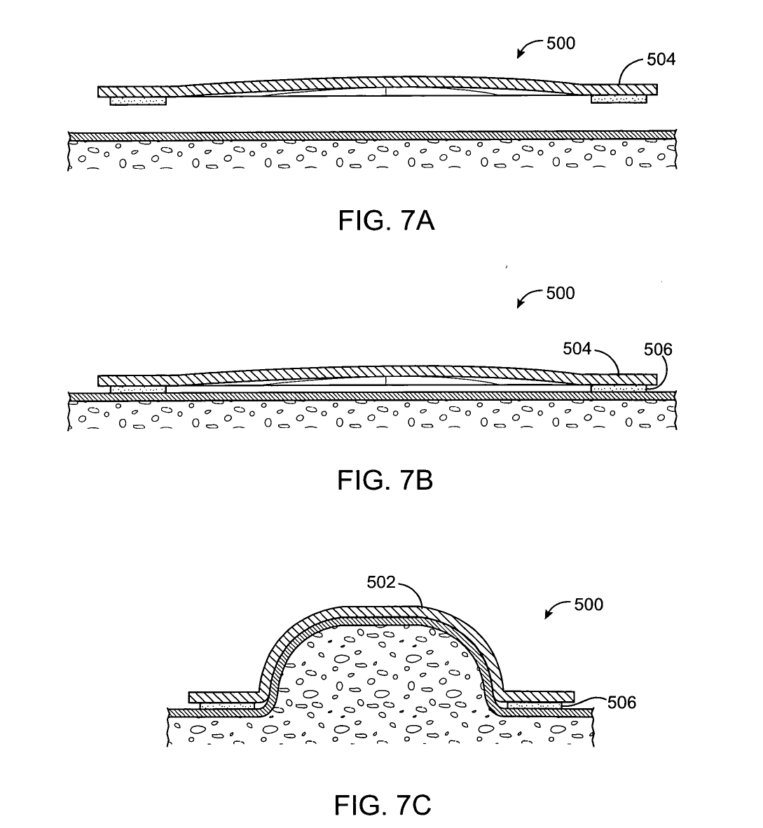

Use of the device 500 is now described. The device 500 is stretched to the expanded state of FIG. 7A and the device 500 is then adhered to the skin and shown in FIG. 7B. The membrane 504 is then released to permit the membrane 504 to constrict toward the relaxed state of FIG. 5A which will cause the excess skin to gather in the pouch 502.

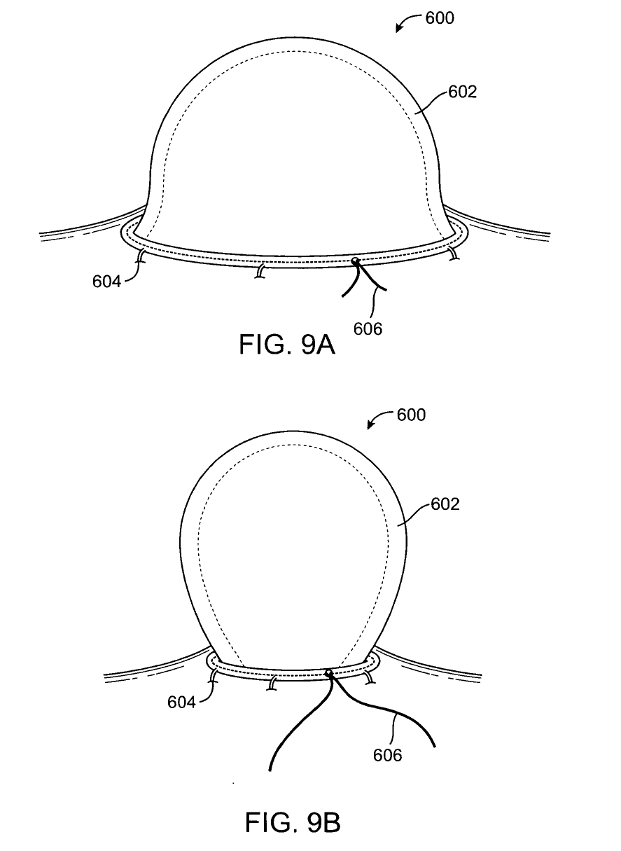

Referring now to FIGS. 9A and 9B, another aspect of the present invention is shown. A support device 600 has a pouch 602 which is attached to the skin with barbs or hooks 604 but may be attached in any other suitable manner. A flexible element 606 may be used to cinch the skin together to gather the bundle of excess skin as shown in FIG. 9B.

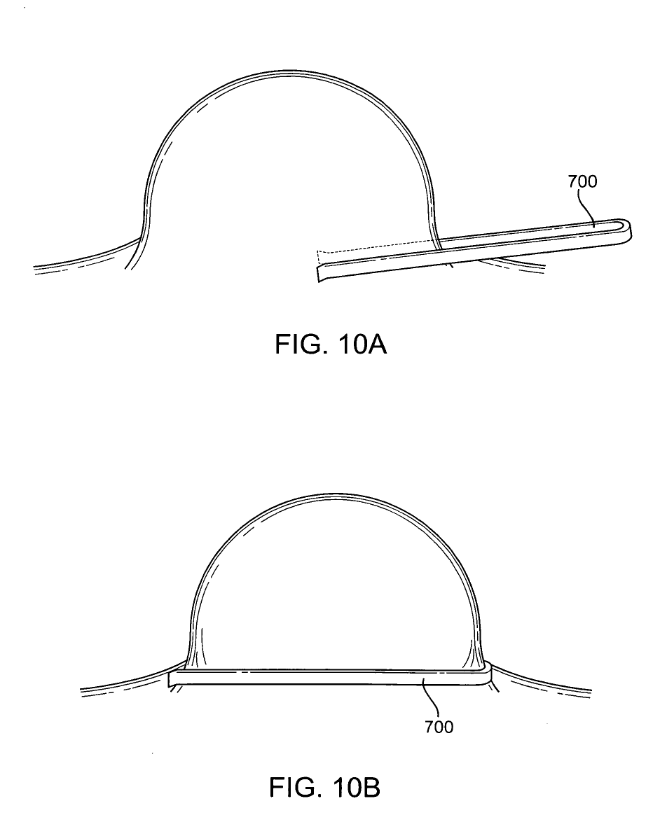

It can be appreciated that numerous other devices and methods for gathering and bundling excess skin may be provided. For example, a clip 700 may be used to gather an excess fold of skin as shown in FIGS. 10A and 10B.

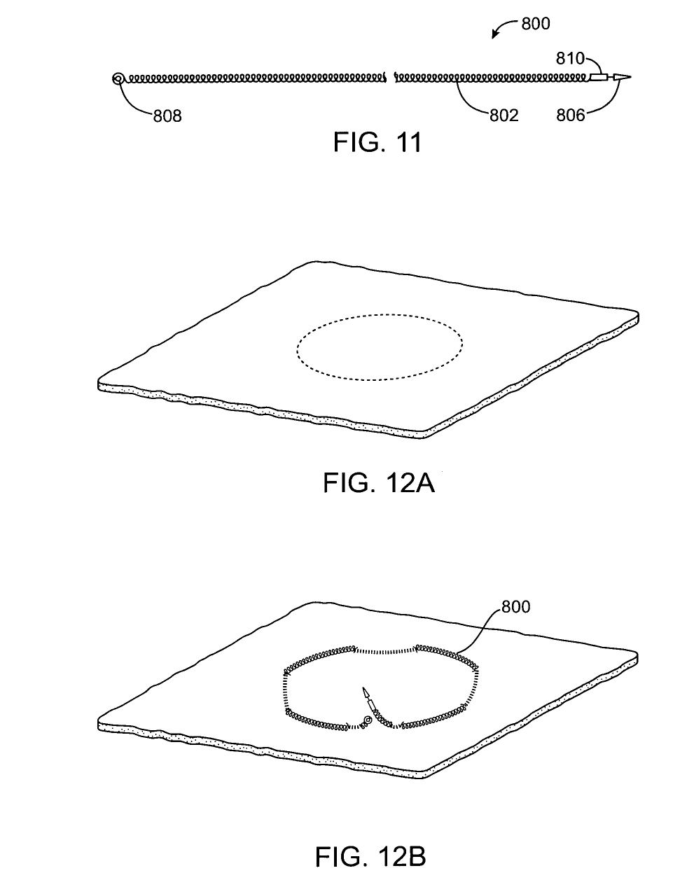

Referring to FIG. 11, still another device 800 for gathering excess skin is shown. The device 800 is formed as a flexible, elongate element 802 of any suitable material. For example, the device 800 may be a nitinol coil having a sharp tip 806. Opposite the sharp tip 806 is a hook 808 which engages a locking element 810 to form a closed loop. The device 800 may be elastic so that it can be stretched to an elongated state.

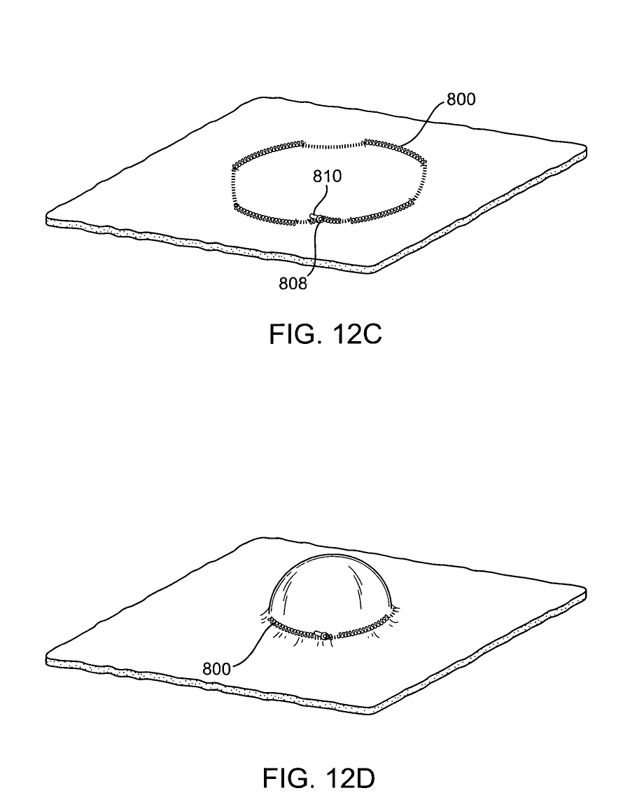

Use of the device 800 is now described. An area of excess skin is identified as the area within the dotted line shown in FIG. 12A. The elongate element 802 is then advanced into the tissue around the area having the excess skin while the element 802 is in a stretched or elongated state as shown in FIG. 12B. The hook 808 is then engaged with the locking element 810 to form a closed loop with the device 800 as shown in FIG. 12C. The element 802 is then released to allow the element 802 to move toward the relaxed or constricted state to assist in gathering and holding the excess tissue as shown in FIG. 12D. The element 802 may be cinched rather than using an elastic response without departing from various aspects of the present invention.

The present invention provides devices and methods for reducing excess skin by inducing the body to absorb the excess skin. The methods and devices are applied to gather and support otherwise normal skin. An advantage of the present invention over surgically removing the excess skin is that the present invention will provide a much smaller scar compared to the scar created by an incision which extends around all of the excess skin. The present invention may be practiced with a relatively small incision large enough to accommodate the dissection tool. The tool may then be used to dissect a relatively large portion of the skin compared to the relatively small incision. The skin is then gathered together and supported to induce the body to absorb the excess skin over time.

The present invention may also find advantages in that the skin may be remodeled and excess skin removed in a number of discrete locations. For example, 4 to 5 discrete areas of excess skin on a breast, separated by untreated areas, may be treated to produce the desired skin remodeling for the entire breast. Depending on the size of the discrete areas and the influence of attachments between the skin and the fascia, remodeling can be achieved without any incision.

The methods and tools taught in this disclosure are applicable to a wide range of excess skin conditions whether resulting from massive weight loss or other conditions. Without limitation, it is anticipated that the methods and tools described may be useful in skin reduction for the legs, arms, abdomen, buttocks, back, neck and face.

What is claimed is:

1. A method for reducing excess skin and remodeling the skin, comprising the steps of:
providing a support device;
selecting an area having excess skin on a body of a patient;
gathering the excess skin into a bundle of excess skin; and
holding the bundle of excess skin with the support device, the bundle of excess skin being held for a period of time sufficient to permit at least a portion of the excess skin to be absorbed by the body.

2. The method of claim 1, wherein:
the holding step is carried out for at least three weeks.

3. The method of claim 1, further comprising the step of:
dissecting the excess skin from an underlying layer before the gathering step.

4. The method of claim 3, wherein:
the dissecting step is carried out by creating an incision and inserting a dissecting tool to dissect the excess skin from the underlying layer.

5. The method of claim 1, wherein:
the gathering step is carried out by securing a support device to the skin and cinching the support device to gather the skin into the bundle of excess skin.

6. The method of claim 1, wherein:
the providing step is carried out with the support device being elastic, the support device having a relaxed state and being expandable to an expanded state;
the gathering step being carried out by stretching the support device to an expanded shape, coupling the support device to the skin while the support device is in the expanded shape, and releasing the support device to permit the support device to move back toward the relaxed state thereby gathering the bundle of excess skin.

7. The method of claim 6, wherein:
the gathering step is carried out with the support device being adhered to the skin while in the expanded state.

8. The method of claim 1, wherein:
the selecting, gathering and holding steps are carried out without removing tissue.

9. The method of claim 1, wherein:
the selecting step is carried out with the area having the excess skin being an otherwise normal healthy area of the skin.

10. The method of claim 1, wherein:
the providing step is carried out with the support device including a elongate element.

11. The method of claim 10, wherein the gathering step includes the steps of:
coupling the elongate element to the skin;
stretching the elongate element to an elongated state; and
permitting the elongate element to relax thereby constricting the skin to isolate the bundle of excess skin.

12. The method of claim 1, wherein:
the providing step is carried out with the support device having a pouch; and
the holding step is carried out with the pouch containing the bundle of excess skin.

13. The method of claim 12, wherein:
the providing step being carried out with the pouch being removable.

14. The method of claim 1, wherein:
the gathering and holding steps are carried out at a plurality of locations.

15. The method of claim 14, wherein:
the gathering and holding steps are carried out at the plurality of locations with the plurality of locations being separated by untreated skin.

16. A method for reducing excess skin and remodeling the skin, comprising the steps of:
providing a support device;
selecting an area having excess skin on a body of a patient;
gathering the excess skin into a bundle of excess skin; and
holding the bundle of excess skin with the support device, the bundle of excess skin being held for a period of time sufficient to permit at least a portion of the excess skin to be absorbed by the body.

17. A method for treating excess skin comprising:
creating an incision;
inserting at least one tool in the incision;
using the tool to create a space between the skin and underlying anatomical structures; and
supporting the excess skin such that it is reabsorbed by the body.

18. The method of claim 17, further comprising the step of:
adjusting the skin so that the skin lies substantially smooth over the underlying anatomical structures.

19. The method of claim 17, wherein:
the supporting step is carried out for at least three weeks.

20. The method of claim 17, wherein:
the supporting step is carried out with the excess skin being mechanically unloaded relative to skin around the excess skin.

21. A device for reducing excess skin and remodeling the skin, comprising:
a support device configured to hold a bundle of excess skin for a period of time sufficient to permit at least a portion of the excess skin to be absorbed by the body.

22. The device of claim 21, wherein:
the support device is configured to hold a bundle of excess skin which has been dissected from an underlying layer using a tool, the tool being introduced through an incision which lies within the bundle of excess skin.

23. The device of claim 21, wherein:
the support is cinched to gather the skin into the bundle of excess skin.

24. The device of claim 21, wherein:
the support device is elastic, the support device having a relaxed state and being expandable to an expanded state, the support device being stretched to the expanded shape and coupled to the skin while the support device is in the expanded shape, the support device being released to permit the support device to move back toward the relaxed state thereby gathering the bundle of excess skin.

25. The device of claim 24, wherein:
the support device is adhered to the skin while in the expanded state.

26. The device of claim 21, wherein:
the support device is configured to induce the body to absorb the excess skin from an otherwise normal healthy area of the skin.

27. The device of claim 21, wherein:
the support device includes an elongate element, the elongate element being stretchable to an elongated state, the elongate element being coupled to the skin while in the elongated state and then being permitted to relax toward a constricted state to isolate the bundle of excess skin.

28. The device of claim 21, wherein:
the support device has a pouch which contains the bundle of excess skin.

29. The device of claim 28, wherein:
the pouch is removable.

30. The device of claim 21, wherein:
a plurality of support devices are used to each gather a bundle of excess tissue, the plurality of support devices being separated by untreated skin.
